(19) United States
(12) Patent Application Publication
(10) Pub. No.: US 2019/0133029 A1
(43) Pub. Date: May 9, 2019

(54) METHOD FOR FACILITATING AN ACTIVE AGENT DISTRIBUTION IN AN AGRICULTURAL CULTIVATION AREA

(71) Applicant: Robert Bosch GmbH, Stuttgart (DE)

(72) Inventors: Damian Koesters, Pfungstadt (DE); Helmut Schomburg, Deufringen (DE); Jochen Fehse, Friolzheim (DE)

(21) Appl. No.: 16/160,474

(22) Filed: Oct. 15, 2018

(30) Foreign Application Priority Data
Nov. 8, 2017 (DE) 102017219804.4

(51) Int. Cl. A01C 21/00; G01N 33/18; G05B 19/406

(52) U.S. Cl. CPC A01C 21/007; A01M 21/043; G05B 19/406; G01N 33/18

(57) ABSTRACT

A method for facilitating an active agent distribution in an agricultural cultivation area, including: measuring at least one chemical or physical property of discharged surface water at a plurality of measuring points in the agricultural cultivation area; transmitting measured data of the chemical or physical property to a control unit; allocating the measured data to a plurality of surface regions of the agricultural cultivation area; determining for the respective surface regions an active agent quantity to be applied as a function of the measured data ascertained for the respective surface regions and as a function of a stored rule; and supplying to an output unit an amount of the respective active agent quantity for the respective surface region.

### RELATED APPLICATION INFORMATION

The present application claims priority to and the benefit of German patent application no. 10 2017 219 804.4, which was filed in Germany on Nov. 8, 2017, the disclosure which is incorporated herein by reference.

### FIELD OF THE INVENTION

The present invention relates to a method, control unit, system and computer readable medium for facilitating an active agent distribution in an agricultural cultivation area.

### BACKGROUND INFORMATION

From the document US 2015/0105984 A1, it is already known to equip an agricultural harvester with a control system in order to evaluate a biomass of the harvested material during the harvesting operation and to derive on such a basis an item of information about the soil of the agricultural cultivation area with the aid of a data map for different soil types.

### SUMMARY OF THE INVENTION

In contrast, the method according to the present invention for facilitating the distribution of an active agent in an agricultural cultivation area has the advantage that information pertaining to the soil quality is easily ascertainable even prior to harvesting, and that an application of an active agent for the improved utilization of the agricultural cultivation area is able to be selectively carried out as a function of a soil quality. In this context, it is advantageously exploited that rainwater impinging upon a ground surface flushes out substances contained in the soil and particles of the soil. In an advantageous manner, a chemical or physical property of the rainwater is now measured by individual sensors that are distributed across the agricultural cultivation area. Using a corresponding rule, by which a soil quality is allocated to the chemical or physical property, an area-dependent quantity of an active agent is determined as a function of the thusly determined soil quality, which is to be applied to a surface region allocated to the measuring sensor that was used to analyze the discharged rainwater in the respective surface region. This makes it is possible to adapt a quantity of an active agent to be applied to the respective soil condition. It can then be ensured that exactly the correct quantity of an active agent is applied, that is to say, neither too little nor too much. An efficient use of an active agent is therefore able to be achieved. Overdosing, which is harmful to the environment, is avoided and also underdosing, which reduces the yield. The respective active agent quantity to be applied is automatically made available at a dispensing unit. The measured data are automatically acquired by a multitude of sensors, thereby obviating the need to collect soil samples, for instance. Instead, utilizing the discharged rainwater, an easily measurable solution or suspension is provided for an analysis, and is supplied to the sensors in an uncomplicated manner.

Additional advantages result from the dependent claims. For example, it is advantageous to add a subsequent distribution step in which a dispensing device determines its position so that information as to which active agent quantity is to be applied to a surface region allocated to this location may be supplied to the dispensing device as a function of the location. On this basis, the dispensing of the active agent may be carried out in an automated manner in such a way that exactly the right dosage of the active agent is applied to the agricultural cultivation area. No manual adjustment of an active agent quantity to be dispensed is required for this purpose. Instead, depending on the respective location position and with access to the supplied active agent quantity provided for a surface region, the correct quantity of the active agent is applied to the agricultural cultivation area.

It is furthermore advantageous to detect a rainfall so that a determination of the physical or chemical property of the discharged rainwater is carried out only during a rainfall or within a predefined period of time following a rainfall. In this way it can be ensured that sufficient rainwater to be discharged is available for the entire agricultural cultivation area and that harmful effects, e.g., by water introduced from some other source or by a solution applied by a farmer himself, are avoided. Effects caused by substances that are possibly added, e.g., during an irrigation process, are thus avoidable.

To begin with, any substances that are able to affect plant growth in the agricultural cultivation area may be utilized as active agents. These may advantageously involve fertilizers because in the case of fertilizers, a precise determination of the applicable fertilizer quantity is particularly advantageous for reasons of efficiency and environmental protection. If the quantity is too small, plants may possibly not grow sufficiently, and if the quantity is too large, the environment is adversely affected and costs arise. In the standard case, the soil of an agricultural cultivation area always already provides a certain measure of growth-promoting substances. The measuring of the chemical or physical properties of the discharged surface water according to the present invention makes it possible to derive a soil quality such that a precise dosing of a fertilizer quantity to be supplied is able to take place. In addition, it is advantageous to provide as an active agent a plant protection agent by which the growth of weeds is able to be restricted or by which pests are able to be controlled. If the soil still holds sufficient quantities of corresponding substances, then the dosing may possibly be reduced.

It is furthermore advantageous to determine as a chemical or physical property a Ph value of the rainwater, a nitrate content, a phosphorus content, a potassium content, and/or a quantity of the discharged rainwater. These quantities advantageously make it possible to derive measures for the quality of the soil. In this context it may be assumed that a comparable soil quality in the presence of a rain quantity causes a corresponding reproduction of a concentration of these substances in the discharged rainwater.

It is furthermore advantageous to carry out the measurement of the chemical or physical properties at measuring points in a drainage line or in a conduit to a drainage line in each case. In the standard case, after the application of surface water or after a rainfall, a sufficient amount of water is available in the drainage line, so that a reliable measurement is possible.

Moreover, it is advantageous to connect a sensor node to a plurality of sensors in such a way that measuring results are more easily able to be transmitted from the sensor node to a central instance, which may be via a radio connection.

Corresponding advantages result for a control unit according to the present invention and for a system for executing the method according to the present invention.

Exemplary embodiments of the present invention are shown in the drawing and are described in greater detail in the following description.

### BRIEF DESCRIPTION OF THE DRAWINGS

FIG. 1 shows an agricultural cultivation area having a plurality of sensors and a control unit for executing the method according to the present invention, as well as a device for applying the active agent.

FIG. 2 shows an exemplary embodiment of a sensor.

FIG. 3 shows an assembly example of sensor devices and a sensor node in a drainage line.

FIG. 4 shows an exemplary embodiment for the execution of a method according to the present invention.

### DETAILED DESCRIPTION

The method for facilitating the distribution of an active agent according to the present invention may be used for various types of active agents and also for checking the success of an application of an active agent; to do so, after an application, the concentration of a substance that is changed due to the application of the active agent is determined in the agricultural cultivation area and the surface region.

More specifically, however, prior to applying the active agent, it is provided to determine the specific need for the active agent in the agricultural cultivation area and to do so by monitoring a chemical or physical property of the surface water discharged from the agricultural cultivation area, the measurement taking place for a specific surface region so that a local determination of an active agent quantity for a specific surface region may be carried out. In the following exemplary embodiment, the present invention will be described for the application of a fertilizer to an agricultural cultivation area.

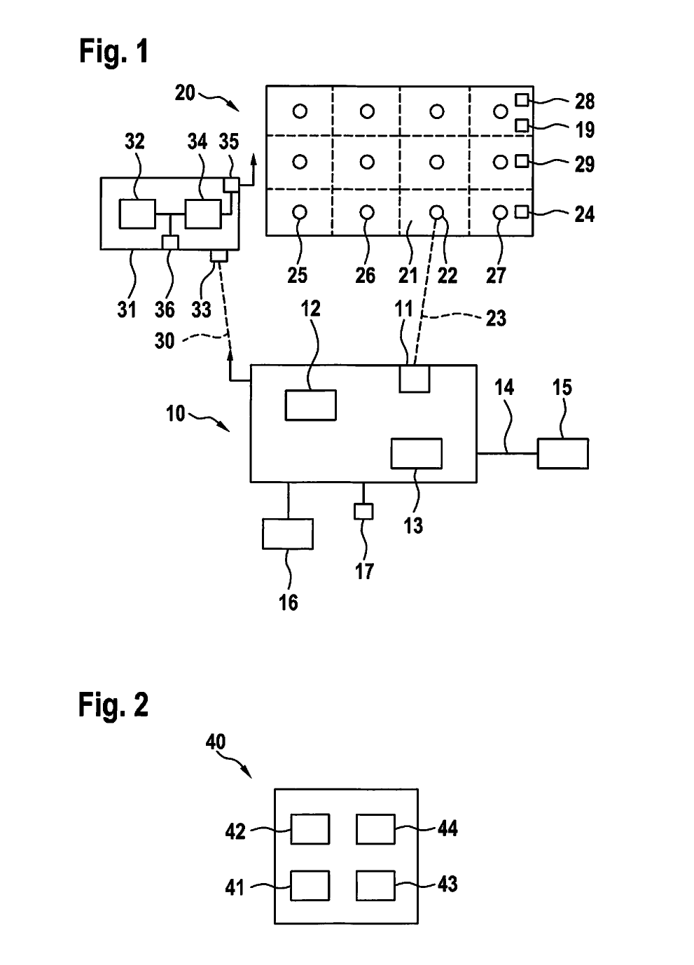

FIG. 1 shows a control unit 10, which is used to supply the amount of a respective fertilizer quantity for surface regions of an agricultural cultivation area 20. Agricultural cultivation area 20 has different surface regions, and a first surface region 21 has been provided with a reference numeral by way of example. In the exemplary embodiment illustrated in this instance, the surface regions have been selected to be equal in size, but they may also have different sizes. For example, a surface region has a size between 10 m×10 m and 100 m×100 m, which may be 25 m×25 m. Each surface region is assigned a sensor, which is used to measure rainwater discharged from the surface region, e.g., first surface region 21, with regard to a chemical or physical property. In a first specific embodiment, the measuring result for first surface region 21 is transmitted from an allocated first sensor 22 via a wireless connection 23 to a receiver unit 11 of control unit 10. In another specific embodiment, it may also be the case that different sensors are connected to a sensor node 24, and sensor node 24 transmits an item of information from first sensor 22 and possibly from additional sensors 25, 26, 27 to receiver unit 11.

A processing unit 12 of control unit 10 evaluates the received data. In addition, an identification of the respective sensor, e.g., of first sensor 22, is also analyzed in the process. Precise location information about the respective surface region is stored in a memory of the respective sensor during an initialization of sensors 22, 25, 26, 27.

The measuring data together with corresponding location information are transmitted to control unit 10, and processing unit 12 evaluates this information by allocating the measuring results to the respective stored surface regions.

The measuring results are compared to information that pertains to a soil quality or to a fertilizer quantity to be applied and that has been stored in a memory unit 13. In a further specific embodiment, it is also possible to set up a corresponding database via a data-network connection 14 to a central instance 15 for assisting in an evaluation. A database containing corresponding empirical values for different measured values is stored in central instance 15 or in memory unit 13, so that an active agent quantity to be applied is able to be derived from the database.

The sensors may be configured for the measurement of nutrients or toxic substances. One or more of the following variable(s) of the discharged surface water may be acquired: the Ph value, the nitrate content (NO₃), phosphorus content (P₂O₅), potassium content (K₂O) and the quantity of the discharged surface water.

In a first specific embodiment, the fertilizer quantity to be applied to a surface region is able to be displayed to a user on a display 16. In a further specific embodiment, it is provided to actuate an agricultural working device 31, e.g., an agricultural work vehicle having a fertilizer-dispensing device 32, in particular via a wireless connection 30 by way of an interface 33. The active agent quantity to be dispensed is transmitted via interface 33 to a processing unit 34 of agricultural work vehicle 31. Agricultural work vehicle 31 also includes a localization unit 35 such as a satellite tracking device. If agricultural work vehicle 31 then drives across agricultural cultivation area 20, localization unit 35 determines the position of agricultural work vehicle 31. Processing unit 34 of agricultural work vehicle 31 allocates the position of one of surface regions 21. Depending on an active agent quantity intended for the individual surface region, and under consideration of the current position and which may also be under consideration of a speed of agricultural work vehicle 31, which is determined via a movement tracker 36 of agricultural work vehicle 31, for example, processing unit 34 controls active agent dispensing unit 32 in such a way that the individually determined predefined active agent quantity is applied inside the agricultural cultivation area in the individual surface regions. In particular, a dosage, i.e., a speed-dependent dispensing of the active agent, is adjusted in such a way that a desired active agent quantity will be dispensed while passing through, in particular at a uniform speed across the corresponding surface region of the agricultural cultivation area. In the process, the individual surface regions may be subdivided into different driving strips while passing through, for instance, and be considered accordingly when the active agent is dispensed. The applied active agent quantity is automatically adapted when a boundary between different surface regions is crossed, while automatically taking the position determined via localization unit 35 into account.

FIG. 2 shows an exemplary embodiment of a sensor 40 for use in agricultural cultivation area 20. Sensor 40 includes a sensor unit 41, which may possibly have a plurality of individual sensors for determining the magnitude of the physical or chemical property to be measured in each case. Different measuring principles may be used for this purpose, e.g., a measurement of the electrical conductivity or a measurement of a Ph-value. In addition, it is also possible to process a collected sample of the surface water in subsequent analyzation steps, using for this purpose a chemical processing unit having what is known as a lab-on-chip unit, in order to determine the chemical or physical property. In addition, it is also advantageous to provide a flow-rate sensor in order to be able to determine a draining quantity of the surface water so that a measured concentration may be related to an available rain or irrigation quantity.

In addition, sensor 40 includes a processing unit 42, so that measuring results from sensor 41 are able to be evaluated. Moreover, an energy-supply unit 43 may be provided, e.g., an energy-accumulator unit or an energy-harvesting unit. In addition, a data interface 44, in particular a radio interface, is provided, which is able to be used to establish a communication with a sensor node 24 and/or control unit 10 so that measuring results of the physical or chemical property of the surface water, measured with the aid of sensor unit 41, are able to be transmitted.

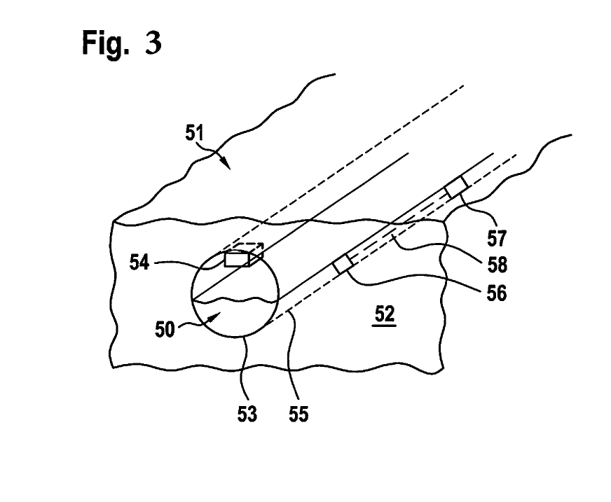

FIG. 3 shows an exemplary embodiment of an assembly of a sensor node and different sensors in a drainage line 50, which runs underneath an agricultural cultivation area 51 in soil 52. A sensor node 54, which transmits detected measured data to control unit 10, is disposed at a water outlet 53 of drainage line 50. In one specific embodiment, it is also already possible to preprocess measured data there. The measured data, together with the positions of the respective sensors, are transmitted to control unit 10, for instance via a cable or via a radio link, which is developed as W-Lan or a mobile telephony connection, for example. In a further specific embodiment, it is also possible to transmit data by way of a Cloud-based system via the internet and to transmit the data to control unit 10 by way of a server connected to the internet. Disposed within the drainage line, in particular at a bottom 55, are a first sensor 56 and a second sensor 57, sensor node 54 being connected to sensors 56, 57 by way of a cable link 58. Sensors 56, 57 are basically developed to be watertight, except for the openings for the measurements. In one specific embodiment, sensors 56, 57 become active when they measure a humidity and then begin transmitting measured data to sensor node 54 at regular time intervals, such as between 10 minutes and an hour. Multiple drainage lines may be situated in parallel in a field, the parallel arrangement making it possible to measure the entire agricultural cultivation area as far as possible. During the initialization of the sensors, the respective position of the sensor is specified in the sensors. When a new drainage system is set up, sensors are able to be distributed across an agricultural cultivation area. In addition, it is also possible to retrofit already existing drainage systems with corresponding sensors.

In a first specific embodiment, control unit 10 may be disposed in a central instance that is located at a distance from the agricultural cultivation area. However, it is additionally also possible to equip a portable computer, e.g., a smartphone, with the functions of the central instance. A control of the system as a whole is implemented via display unit 16 and also via an input unit 17 of control unit 10. In a further specific embodiment, it is also possible to integrate control unit 10 into agricultural work vehicle 31.

A rain sensor 28 is provided, in particular at an edge of the agricultural cultivation area, which determines whether a minimum quantity of rain is exceeded during a rainfall. In the affirmative case, it activates a measurement through an actuation of sensor nodes 19, 24, 29, which in turn instruct the individual sensors disposed in agricultural cultivation area 20 to carry out a measurement. In this way it can be avoided that energy will be expended by the sensors for a measurement during a dry phase when no surface water is available for a measurement. In a corresponding manner, a measurement may also be triggered during an irrigation process.

In addition, the measured rain quantity may also be used to draw conclusions with regard to a total supplied rain quantity, thereby allowing the detected physical or chemical quantity to be evaluated with regard to the total supplied water quantity. Effects caused by the water quantity, such as a reduced concentration of dissolved substances, are thereby able to be properly taken into account.

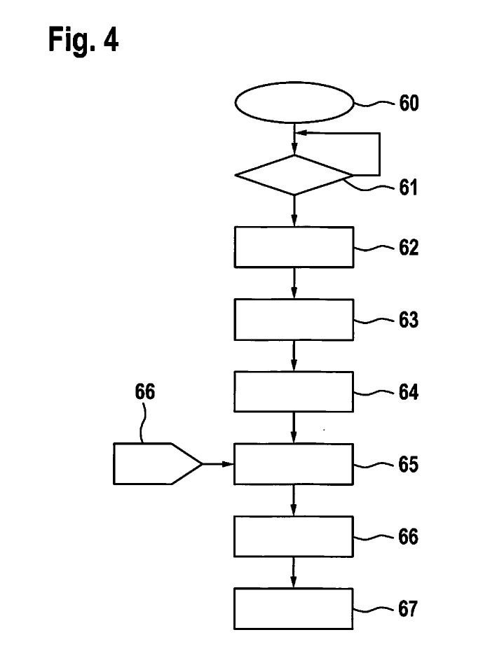

FIG. 4 shows an exemplary embodiment of a method according to the present invention. In an initialization step 60, the sensors are registered in control unit 10 and the measuring method is initialized. In a check step 61, it is queried whether surface water, in particular as a result of rain, is available. If this is not the case, then it is branched back. If this is indeed the case, then branching to a measuring step 62 is carried out in which the measured data of the individual sensors are ascertained with regard to the physical or chemical property of the surface water. In a subsequent transmission step 63, the measured data are transmitted to the control unit. In an allocation step 64, the measured data are allocated to individual surface regions of the agricultural cultivation area. In a determination step 65, a quantity of an active agent to be applied to a respective surface region is determined for each surface region while taking empirical values 66 into account. The quantity of the active agent for each surface region is then made available in a supply step. In a dispensing step 67, the quantity of the active agent is applied to the respective surface region as a function of the location.

What is claimed is:

1. A method for facilitating an active agent distribution in an agricultural cultivation area, the method comprising:
measuring at least one chemical or physical property of discharged surface water at a plurality of measuring points in the agricultural cultivation area;
transmitting measured data of the chemical or physical property to a control unit;
allocating the measured data to a plurality of surface regions of the agricultural cultivation area;
determining for the respective surface regions a respective active agent quantity to be applied as a function of the measured data ascertained for the respective surface regions and as a function of a stored rule; and
supplying an amount of the respective active agent quantity for the respective surface region at an output unit.

2. The method of claim 1, further comprising:
distributing the active agent in the agricultural cultivation area, including an outputting of an amount of the respective active agent quantity for the respective fertilizer quantity for the respective surface region to a dispensing device for the active agent;
determining a position of the dispensing device in the agricultural cultivation area;
wherein the active agent is controlled as a function of the determined position of the dispensing device in the agricultural cultivation area so that the determined quantity of the active agent intended for a surface region in which the detected position is located is distributed to the surface region.

3. The method of claim 1, further comprising:
determining a rainfall or an irrigation event, in which a measurement of the at least one physical or chemical property of the discharged surface water is carried out only during the rainfall or the irrigation event or within a predefined time period following the rainfall or the irrigation event.

4. The method of claim 1, further comprising:
applying a plant fertilizer as an active agent to the agricultural cultivation area.

5. The method of claim 1, wherein a pH value, a nitrate content, a phosphorus content, a potassium content and/or a quantity of the discharged rainwater is/are determined as the chemical or physical property.

6. The method of claim 1, wherein the measurement of the chemical or physical property is carried out at measuring points in a drainage line or in a conduit to a drainage line.

7. The method of claim 1, wherein measured data are transmitted from a sensor node to the control unit for a determination of the active agent quantity, and a plurality of sensors is connected to the sensor node in each case.

8. The method of claim 1, wherein the measured data are transmitted in a wireless manner.

9. A control unit, comprising:
an interface;
a plurality of sensors for determining a chemical or physical property of discharged rainwater; and
a controller for facilitating an active agent distribution in an agricultural cultivation area, and configured to perform the following:
measuring at least one chemical or physical property of discharged surface water at a plurality of measuring points in the agricultural cultivation area;
transmitting measured data of the chemical or physical property to a control unit;
allocating the measured data to a plurality of surface regions of the agricultural cultivation area;
determining for the respective surface regions a respective active agent quantity to be applied as a function of the measured data ascertained for the respective surface regions and as a function of a stored rule; and
supplying an amount of the respective active agent quantity for the respective surface region at an output unit.

10. A system, comprising:
a control unit, including:
an interface;
a plurality of sensors for determining a chemical or physical property of discharged rainwater; and
a controller for facilitating an active agent distribution in an agricultural cultivation area, and configured to perform the following:
measuring at least one chemical or physical property of discharged surface water at a plurality of measuring points in the agricultural cultivation area;
transmitting measured data of the chemical or physical property to a control unit;
allocating the measured data to a plurality of surface regions of the agricultural cultivation area;
determining for the respective surface regions a respective active agent quantity to be applied as a function of the measured data ascertained for the respective surface regions and as a function of a stored rule; and
supplying an amount of the respective active agent quantity for the respective surface region at an output unit and a central instance.

11. A non-transitory computer readable medium having a computer program, which is executable by a processor, comprising:
a program code arrangement having program code for facilitating an active agent distribution in an agricultural cultivation area, by performing the following:
measuring at least one chemical or physical property of discharged surface water at a plurality of measuring points in the agricultural cultivation area;
transmitting measured data of the chemical or physical property to a control unit;
allocating the measured data to a plurality of surface regions of the agricultural cultivation area;
determining for the respective surface regions a respective active agent quantity to be applied as a function of the measured data ascertained for the respective surface regions and as a function of a stored rule; and
supplying an amount of the respective active agent quantity for the respective surface region at an output unit and a central instance.

12. The computer readable medium of claim 11, further comprising:
distributing the active agent in the agricultural cultivation area, including an outputting of an amount of the respective active agent quantity for the respective fertilizer quantity for the respective surface region to a dispensing device for the active agent;
determining a position of the dispensing device in the agricultural cultivation area;
wherein the active agent is controlled as a function of the determined position of the dispensing device in the agricultural cultivation area so that the determined quantity of the active agent intended for a surface region in which the detected position is located is distributed to the surface region.
